(19) United States
(12) Patent Application Publication
(10) Pub. No.: US 2024/0350798 A1
(43) Pub. Date: Oct. 24, 2024

(54) NEUROSTIMULATION SYSTEMS WITH STIMULATION LEADS AND/OR STYLETS WITH IMPROVED MALLEABILITY AND/OR FLEXIBILITY

(71) Applicant: Advanced Neuromodulation Systems, Inc., Plano, TX (US)

(72) Inventors: Aaron Raines, Dallas, TX (US); Richard Ornelaz, Frisco, TX (US); Wei Carrigan, Grapevine, TX (US)

(21) Appl. No.: 18/644,071

(22) Filed: Apr. 23, 2024

Related U.S. Application Data
(60) Provisional application No. 63/461,585, filed on Apr. 24, 2023.

(51) Int. Cl. A61N 1/05

(52) U.S. Cl. CPC A61N 1/0553

(57) ABSTRACT

The present application generally relates to neurostimulation systems with stimulation leads and/or stylets with improved malleability and/or flexibility.

### CROSS-REFERENCE TO RELATED APPLICATIONS

The present application claims the benefit of priority from U.S. Provisional Application No. 63/461,585 filed Apr. 24, 2023 and entitled “NEUROSTIMULATION SYSTEMS WITH STIMULATION LEADS AND/OR STYLETS WITH IMPROVED MALLEABILITY AND/OR FLEXIBILITY,” the disclosure of which is incorporated by reference herein in its entirety.

### TECHNICAL FIELD

The present disclosure generally relates to neurostimulation systems with stimulation leads and/or stylets with improved malleability and/or flexibility.

### BACKGROUND

Implantable medical devices are used for a wide variety of medical conditions. For example, a number of implantable medical devices have been commercially distributed that allow electrical pulses or signals to be controllably delivered to targeted tissue or nerves after implantation of the respective device within a patient. Such implantable medical devices may be used for cardiac pace making, cardiac rhythm management, treatments for congestive heart failure, implanted defibrillators, and neurostimulation. Neurostimulation encompasses a wide range of applications, such as for example, treatment of chronic pain, treatment of motor disorders, treatment of incontinence and other sacral nerve related disorders, reduction of epileptic seizures, and treatment of depression.

Neurostimulation in the form of spinal cord stimulation (SCS), for example, has been used as a treatment for chronic pain for a number of years. SCS is often used to alleviate pain after failed surgery, pain due to neuropathies, or pain due to inadequate blood flow. In accordance with SCS therapy, non-nociceptive fibers are stimulated to alleviate pain symptoms in cases of chronic pain.

Implantable electrical stimulation devices generally include an implanted pulse generator that generates electrical pulses or signals that are transmitted to targeted tissue or nerves through a therapy delivery element, such as a lead with an electrode array. In the case of SCS, an electrode array present on a distal end of a lead may be implanted so as to be disposed within the epidural space for delivery of the electrical stimulation. A pulse generator coupled to a proximal end of the lead may thus be enabled to apply neural stimuli to the dorsal column in order to give rise to a compound action potential (CAP). The dorsal column contains the afferent A-beta (Ab) fibers to mediate sensations of touch, vibration, and pressure from the skin, whereby ones of the Ab fibers may be therapeutically recruited by the neural stimuli provided through the electrode array by the pulse generator.

According to conventional SCS, stimulation pulses are provided to neural tissue of the dorsal column in a regular pattern with each pulse having a predetermined amplitude (e.g., current intensity) and being separated by a fixed inter-pulse interval that defines a stimulation frequency configured for inducing a tingling sensation (known medically as paresthesia) in the patient. For example, stimulation of the Ab fibers may induce paresthesia and therefore may provide the mechanism of action for traditional tonic SCS to mask the pain. Although the paresthesia can be uncomfortable or even painful in patients, the paresthesia is often substantially more tolerable than the pain otherwise experienced by the patients.

Another approach to pain management through SCS uses a stimulation technique called burst stimulation. In implementation of burst stimulation therapy, packets (e.g., “bursts”) of high-frequency impulses are delivered periodically (e.g., five pulses at 500 Hz, delivered 40 times per second) at a current intensity below the paresthesia threshold. It has been found that such burst stimulation suppresses neuropathic pain at least as well as, and possibly better than, traditional tonic SCS stimulation and provides such pain relief without eliciting paresthesia. Burst stimulation that bypasses the paresthesia process is hypothesized to have a different mechanism of action than that of traditional tonic SCS stimulation, and therefore may bypass Ab fiber activation (see e.g., Arie et al., “High frequency stimulation of dorsal column axons: potential underlying mechanism of paresthesia-free neuropathic pain”, incorporated by reference above; Beurrier, et al., “Subthalamic nucleus neurons switch from single-spike activity to burst-firing mode,” J. Neurosci., 19 (2): 599-609, 1999; and Stefan Schu, MD, PHD, Philipp J. Slotty, MD, Gregor Bara, MD, Monika von Knop, Deborah Edgar, PhDt, Jan Vesper, MD, PHD, “A Prospective, Randomised, Double-blind, Placebo-controlled Study to Examine the Effectiveness of Burst Spinal Cord Stimulation Patterns for the Treatment of Failed Back Surgery Syndrome”, Neuromodulation 2014; 17:443-450; the disclosures of which are incorporated herein by reference).

An additional approach to pain management through SCS is to use high-frequency SCS (HFSCS) to provide paresthesia-free therapy. HFSCS typically includes pulses at frequencies between 1500 Hz and 10,000 Hz although even higher frequencies could be used. In accordance with HFSCS, high-frequency electrical pulses are delivered at a current intensity below the paresthesia threshold. For example, HFSCS stimulation regimens implementing a stimulation frequency of up to 10 kHz have been found to be effective in providing pain relief without eliciting paresthesia (see e.g., Arie J E, Mei L, Carlson K W, and Shils J L, “High frequency stimulation of dorsal column axons: potential underlying mechanism of paresthesia-free neuropathic pain”, Poster at International Neuromodulation Society Conference, 2015; and Adnan Al-Kaisy, MD, Jean-Pierre Van Buyten, MD, Iris Smet, MD, Stefano Palmisani, MD, David Pang, MD, and Thomas Smith, MD, “Sustained Effectiveness of 10 kHz High-Frequency Spinal Cord Stimulation for Patients with Chronic, Low Back Pain: 24-Month Results of a Prospective Multicenter Study”, Pain Medicine, 2014, 15:347-354; the disclosures of which are incorporated herein by reference).

Irrespective of the particular SCS stimulation technique implemented, stimuli amplitude (e.g., current intensity) and/or delivered charge are conventionally maintained below a comfort threshold (above which recruitment of Ab fibers may be at a level so large as to produce discomfort and even pain in the patient) in order to provide comfortable operation for a patient. Correspondingly, stimuli amplitude and/or delivered charge are generally maintained above a recruitment threshold to recruit desired action potentials for providing effective therapy to the patient (e.g., inducing an analgesic effect whereby the patient experiences no pain, or a relatively small amount of pain, at the region of interest).

### BRIEF DESCRIPTION OF THE DRAWINGS

FIG. 1 depicts a neurostimulation system according to some representative embodiments.

FIG. 2 depicts a computing device that may be included within a neurostimulation system or to communicate with a neurostimulation system according to some representative embodiments.

FIG. 3 depicts a network environment for remote management of patient care according to some representative embodiments.

FIG. 4 depicts an implantable pulse generator with convention stimulation leads.

FIG. 5 depicts an implantable pulse generator with stimulation leads according to some representative embodiments.

FIG. 6 depicts a stimulation lead according to some representative embodiments.

FIG. 7 depicts an implantable pulse generator and stimulation leads according to some representative embodiments.

FIGS. 8-10 depict multi-stranded stylets according to some representative embodiments.

### DETAILED DESCRIPTION

Neurostimulation systems are devices that generate electrical pulses and deliver the pulses to neural tissue of a patient to treat a variety of disorders. One category of neurostimulation systems is deep brain stimulation (DBS). In DBS, pulses of electrical current are delivered to target regions of a subject's brain, for example, for the treatment of movement and effective disorders such as PD and essential tremor. Another category of neurostimulation systems is spinal cord stimulation (SCS) which is often used to treat chronic pain such as Failed Back Surgery Syndrome (FBSS) and Complex Regional Pain Syndrome (CRPS). SCS devices may also treat a number of other disorders in addition to chronic pain. Dorsal root ganglion (DRG) stimulation is another example of a neurostimulation therapy in which electrical stimulation is provided to the dorsal root ganglion structure that is just outside of the epidural space. DRG stimulation is also generally used to treat chronic pain but may treat other disorders. Neurostimulation therapies including SCS stimulation and DRG stimulation are also known to effect other physiological processes such as cardiac, respiratory, and digestive processes as examples.

Neurostimulation systems generally include a pulse generator and one or more stimulation leads. A stimulation lead includes a lead body of insulative material that encloses wire conductors. The distal end of the stimulation lead includes multiple electrodes, or electrical contacts, that intimately impinge upon patient tissue and are electrically coupled to the wire conductors. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or “can”) that encloses circuitry for generating the electrical stimulation pulses, control circuitry, communication circuitry, a rechargeable or primary cell battery, etc. The pulse generating circuitry is coupled to one or more stimulation leads through electrical connections provided in a “header” of the pulse generator. Specifically, feedthrough wires typically exit the metallic housing and enter into a header structure formed of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

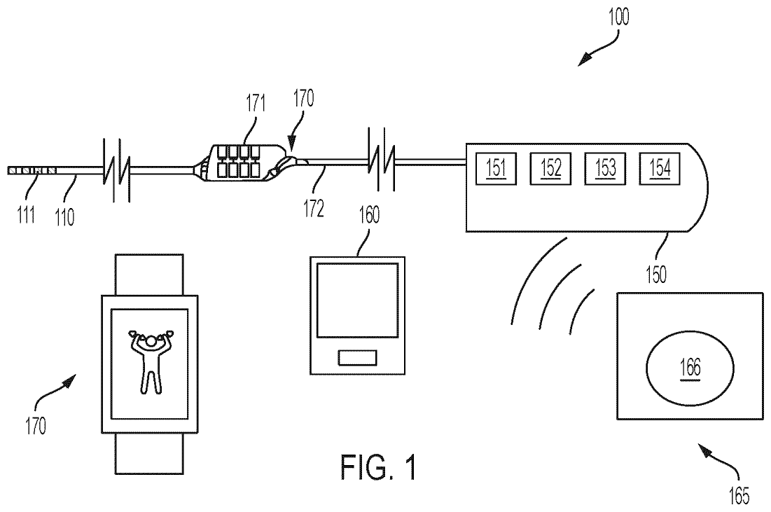

Stimulation system 100 is shown in FIG. 1 according to some embodiments. Stimulation system 100 generates electrical pulses for application to tissue of a patient to treat one or more disorders of the patient. System 100 includes an implantable pulse generator (IPG) 150 that is adapted to generate electrical pulses for application to tissue of a patient. Examples of commercially available implantable pulse generators include the PROCLAIM XR™ and INFINITY™ implantable pulse generators (available from ABBOTT, PLANO Tex.). Alternatively, system 100 may include an external pulse generator (EPG) positioned outside the patient's body. IPG 150 typically includes a metallic housing (or can) that encloses a controller 151, pulse generating circuitry 152, a battery 153, far-field and/or near field communication circuitry 154 (e.g., BLUETOOTH communication circuitry), and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may comprise one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, metal ribbons, traces, lines, or the like (not shown) from the internal circuitry of pulse generator 150 to output connectors (not shown) of pulse generator 150 which are typically contained in the “header” structure of pulse generator 150. Commercially available ring/spring electrical connectors are frequently employed for output connectors of pulse generators (e.g., “Bal-Seal” connectors). The terminals of one or more stimulation leads 110 are inserted within connector portion 171 for electrical connection with respective connectors or directly within the header structure of pulse generator 150. Thereby, the pulses originating from IPG 150 are conducted to electrodes 111 through wires contained within the lead body of lead 110. The electrical pulses are applied to tissue of a patient via electrodes 111.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within IPG 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

External controller device 160 is a device that permits the operations of IPG 150 to be controlled by a user after IPG 150 is implanted within a patient. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. The interface functionality of controller device 160 is implemented using suitable software code for interacting with the user and using the wireless communication capabilities to conduct communications with IPG 150. One or more user interface screens may be provided in software to allow the patient and/or the patient's clinician to control operations of IPG 150 using controller device 160. In some embodiments, commercially available devices such as APPLE IOS devices are adapted for use as controller device 160 by include one or more “apps” that communicate with IPG 150 using, for example, BLUETOOTH communication.

Controller device 160 preferably provides one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc.

Controller device 160 may permit programming of IPG 150 to provide a number of different stimulation patterns or therapies to the patient as appropriate for a given patient and/or disorder. Examples of different stimulation therapies include conventional tonic stimulation (continuous train of stimulation pulses at a fixed rate), BurstDR stimulation (burst of pulses repeated at a high rate interspersed with quiescent periods with or without duty cycling), “high frequency” stimulation (e.g., a continuous train of stimulation pulses at 10,000 Hz), noise stimulation (series of stimulation pulses with randomized pulse characteristics such as pulse amplitude to achieve a desired frequency domain profile). Any suitable stimulation pattern or combination thereof can be provided by IPG 150 according to some embodiments. Controller device 160 communicates the stimulation parameters and/or a series of pulse characteristics defining the pulse series to be applied to the patient to IPG 150 to generate the desired stimulation therapy.

Examples of suitable therapies include tonic stimulation (in which a fixed frequency pulse train) is generated, burst stimulation (in which bursts of multiple high frequency pulses) are generated which in turn are separated by quiescent periods, “high frequency” stimulation, multi-frequency stimulation, and noise stimulation. Descriptions of respective neurostimulation therapies are provided in the following publications: (1) Schu S., Slotty P. J., Bara G., von Knop M., Edgar D., Vesper J. A Prospective, Randomised, Double-blind, Placebo-controlled Study to Examine the Effectiveness of Burst Spinal Cord Stimulation Patterns for the Treatment of Failed Back Surgery Syndrome. Neuromodulation 2014; 17:443-450; (2) Al-Kaisy Al, Van Buyten J P, Smet I, Palmisani S, Pang D, Smith T. 2014. Sustained effectiveness of 10 kHz high-frequency spinal cord stimulation for patients with chronic, low back pain: 24-month results of a prospective multicenter study. Pain Med. 2014 March; 15 (3): 347-54; and (3) Sweet, Badjatiya, Tan D I, Miller. Paresthesia-Free High-Density Spinal Cord Stimulation for Postlaminectomy Syndrome in a Prescreened Population: A Prospective Case Series. Neuromodulation. 2016 April; 19 (3): 260-7. Noise stimulation is described in U.S. Pat. No. 8,682,44162. Burst stimulation is described in U.S. Pat. No. 8,224,453 and U.S. Published application No. 20060095088. A “coordinated reset” pulse pattern is applied to neuronal subpopulation/target sites to desynchronize neural activity in the subpopulations. Coordinated reset stimulation is described, for example, by Peter A. Tass et al in COORDINATED RESET HAS SUSTAINED AFTER EFFECTS IN PARKINSONIAN MONKEYS, Annals of Neurology, Volume 72, Issue 5, pages 816-820, November 2012, which is incorporated herein by reference. The electrical pulses in a coordinated reset pattern are generated in bursts of pulses with respective bursts being applied to tissue of the patient using different electrodes in a time-offset manner. The time-offset is selected such that the phase of the neural-subpopulations are reset in a substantially equidistant phase-offset manner. By resetting neuronal subpopulations in this manner, the population will transition to a desynchronized state by the interconnectivity between the neurons in the overall neuronal population. All of these references are incorporated herein by reference.

For implementation of the components within IMD 14, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION” which is incorporated herein by reference.

IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

External charger device 165 may be provided to recharge battery 153 of IPG 150 according to some embodiments when IPG 150 includes a rechargeable battery. External charger device 165 comprises a power source and electrical circuitry (not shown) to drive current through coil 166. The patient places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. In operation during a charging session, external charger device 165 generates an AC-signal to drive current through coil 166 at a suitable frequency. Assuming that primary coil 166 and secondary coil are suitably positioned relative to each other, the secondary coil is disposed within the magnetic field generated by the current driven through primary coil 166. Current is then induced by a magnetic field in the secondary coil. The current induced in the coil of the implantable pulse generator is rectified and regulated to recharge the battery of IPG 150. IPG 150 may also communicate status messages to external charging device 165 during charging operations to control charging operations. For example, IPG 150 may communicate the coupling status, charging status, charge completion status, etc.

System 100 may include external wearable device 170 such as a smartwatch or health monitor device. Wearable device may be implemented using commercially available devices such as FITBIT VERSA SMARTWATCH™, SAMSUNG GALAXY SMARTWATCH™, and APPLE WATCH™ devices with one or more apps or appropriate software to interact with IPG 150 and/or controller device 160. In some embodiments, wearable device 170, controller device 160, and IPG 150 conduct communications using BLUETOOTH communications.

Wearable device 170 monitors activities of the patient and/or senses physiological signals. Wearable device 170 may track physical activity and/or patient movement through accelerometers. Wearable device 170 may monitory body temperature, heart rate, electrocardiogram activity, blood oxygen saturation, and/or the like. Wearable device 170 may monitor sleep quality or any other relevant health related activity.

Wearable device 170 may provide one or more user interface screens to permit the patient to control or otherwise interact with IPG 150. For example, the patient may increase or decrease stimulation amplitude, change stimulation programs, turn stimulation on or off, and/or the like using wearable device 170. Also, the patient may check the battery status of other implant status information using wearable device 170.

Wearable device 170 may include one or more interface screens to receive patient input. In some embodiments, wearable device 170 and/or controller device 160 are implemented (individually or in combination) to provide an electronic patient diary function. The patient diary function permits the patient to record on an ongoing basis the health status of the patient and the effectiveness of the therapy for the patient. In some embodiments as discussed herein, wearable device 170 and/or controller device 160 enable the user to indicate the current activity of the patient, the beginning of an activity, the completion of an activity, the case or quality of patient's experience with a specific activity, the patient's experience of pain, the patient's experience of relief from pain by the stimulation, or any other relevant indication of patient health by the patient.

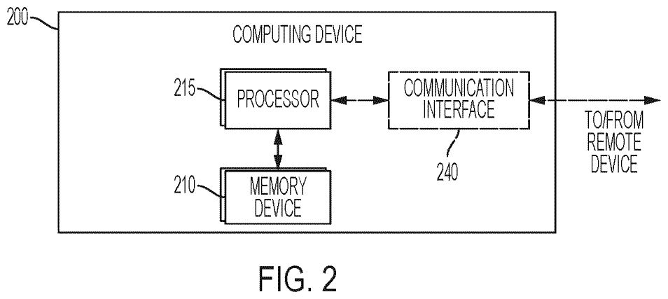

FIG. 2 is a block diagram of one embodiment of a computing device 200 that may be used to according to some embodiments. Computing device 200 may be used to implement external controller device 160, wearable device 170, remote care management servers, or other computing system according to some embodiments.

Computing device 200 includes at least one memory device 210 and a processor 215 that is coupled to memory device 210 for executing instructions. In some embodiments, executable instructions are stored in memory device 210. In some embodiments, computing device 200 performs one or more operations described herein by programming processor 215. For example, processor 215 may be programmed by encoding an operation as one or more executable instructions and by providing the executable instructions in memory device 210.

Processor 215 may include one or more processing units (e.g., in a multi-core configuration). Further, processor 215 may be implemented using one or more heterogeneous processor systems in which a main processor is present with secondary processors on a single chip. In another illustrative example, processor 215 may be a symmetric multi-processor system containing multiple processors of the same type. Further, processor 215 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 210 is one or more devices that enable information such as executable instructions and/or other data to be stored and retrieved. Memory device 210 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 210 may be configured to store, without limitation, application source code, application object code, source code portions of interest, object code portions of interest, configuration data, execution events and/or any other type of data.

Computing device 200, in the illustrated embodiment, includes a communication interface 240 coupled to processor 215. Communication interface 240 communicates with one or more remote devices, such as a clinician or patient programmer. To communicate with remote devices, communication interface 240 may include, for example, a wired network adapter, a wireless network adapter, a radio-frequency (RF) adapter, and/or a mobile telecommunications adapter.

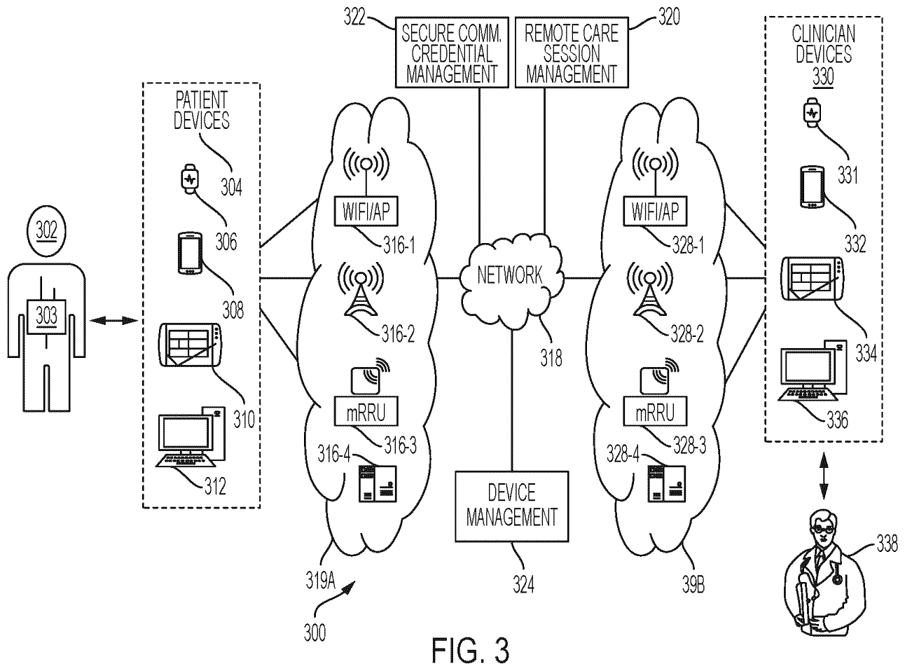

FIG. 3 depicts a network environment 300 for remote management of patient care. One or more embodiments of a remote care therapy application or service may be implemented in network environment 300, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 300 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 302, and one or more authorized clinicians, healthcare professionals, or agents thereof, e.g., generally represented as caregiver(s) or clinician(s) 338.

Example patient(s) 302, each having a suitable implantable device 303, may be provided with a variety of corresponding external devices for controlling, programming, otherwise (re) configuring the functionality of respective implantable medical device(s) 303, as is known in the art. Such external devices associated with patient(s) 302 are referred to herein as patient devices 304, and may include a variety of user equipment (UE) devices, tethered or untethered, that may be configured to engage in remote care therapy sessions. By way of example, patient devices 304 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 306, smartphone(s) 308, tablet(s)/phablet(s) 310 and computer(s) 312. As such, patient devices 304 may include various types of communications circuitry or interfaces to effectuate wired or wireless communications, short-range and long-range radio frequency (RF) communications, magnetic field communications, Bluetooth communications, etc., using any combination of technologies, protocols, and the like, with external networked elements and/or respective implantable medical devices 303 corresponding to patient(s) 302.

With respect to networked communications, patient devices 304 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (SGPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 104 may also include interface circuitry for effectuating network connectivity via satellite communications. Where tethered UE devices are provided as patient devices 304, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 316-1, macro/microcell node(s) 116-2 and 116-3 (e.g., including micro remote radio units or RRUs, base stations, eNB nodes, etc.) and DSL/CMTS node(s) 316-4.

Similarly, clinicians 338 may be provided with a variety of external devices for controlling, programming, otherwise (re) configuring or providing therapy operations with respect to one or more patients 302 mediated via respective implantable medical device(s) 303, in a local therapy session and/or remote therapy session, depending on implementation and use case scenarios. External devices associated with clinicians 338, referred to herein as clinician devices 330, may include a variety of UE devices, tethered or untethered, similar to patient devices 304, which may be configured to engage in remote care therapy sessions as will be set forth in detail further below. Clinician devices 330 may therefore also include devices (which may operate in association with one or more virtual assistants, smart home/office appliances, VRAR virtual reality (VR) or augmented reality (AR) devices, and the like), generally exemplified by wearable device(s) 331, smartphone(s) 332, tablet(s)/phablet(s) 334 and computer(s) 336. Further, example clinician devices 330 may also include various types of network communications circuitry or interfaces similar to that of patient device 304, which may be configured to operate with a broad range of technologies as set forth above. Accordingly, an edge/access network portion 319B is exemplified as having elements such as WiFi/AP node(s) 328-1, macro/microcell node(s) 328-2 and 328-3 (e.g., including micro remote radio units or RRUs, base stations, eNB nodes, etc.) and DSL/CMTS node(s) 328-4. It should therefore be appreciated that edge/access network portions 319A, 319B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 338 and one or more patients 302, wherein such elements are hosted or otherwise operated by various stakeholders in a service deployment scenario depending on implementation (e.g., including one or more public clouds, private clouds, or any combination thereof). In one embodiment, a remote care session management node 320 is provided, and may be disposed as a cloud-based element coupled to network 318, that is operative in association with a secure communications credentials management node 322 and a device management node 324, to effectuate a trust-based communications overlay/tunneled infrastructure in network environment 300 whereby a clinician may advantageously engage in a remote care therapy session with a patient.

U.S. Pat. No. 10,124,177 discloses a system for conducting a remote programming session for an implantable medical device of patient where the clinician operates a clinician programmer at a site that is remote from the location of the patient. U.S. Pat. No. 10,124,177 is incorporated herein by reference.

In the embodiments described herein, implantable medical device 303 may be any suitable medical device. For example, implantable medical device may be a neurostimulation device that generates electrical pulses and delivers the pulses to nervous tissue of a patient to treat a variety of disorders.

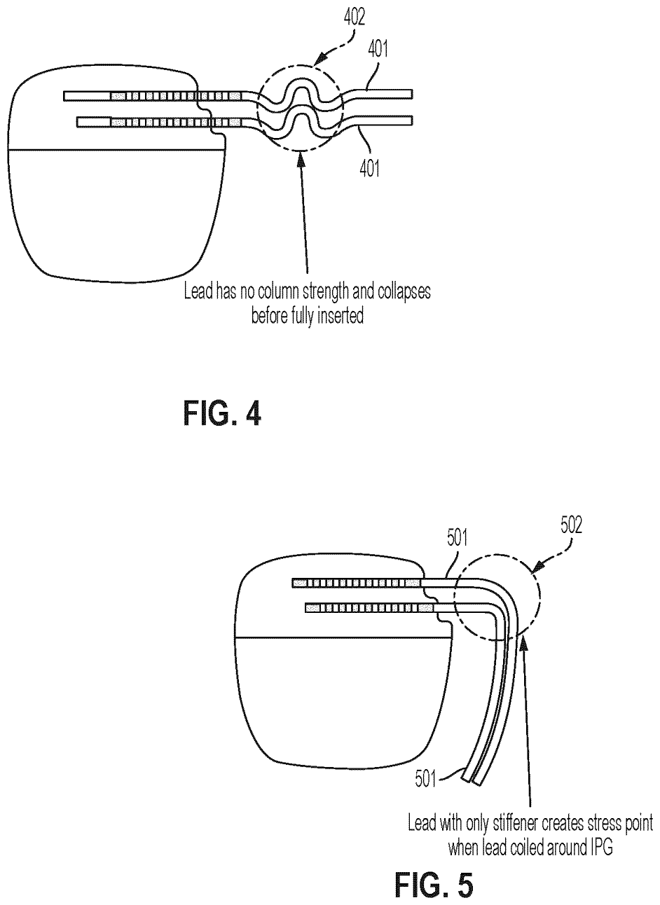

As previously discussed, IPG 150 may include a “header structure” to receive one or more stimulation leads or lead extensions. The proximal end of a stimulation lead or lead extension typically includes one or more “terminals” adapted to electrically contact corresponding structures (e.g., “Bal-Seal” components) within the header. The lead body of stimulation leads are typically relatively flexible to assist the implant procedure and for other reasons related to long-term implantation. The proximal end of a stimulation lead (see, e.g., leads 401 in FIG. 4) is also designed such that the proximal end is also flexible and exhibits minimal column strength. As a result, the stimulation lead will collapse when a clinician attempts to insert the stimulation lead within the header of the IPG, as shown at region 402 in FIG. 4. In such circumstances, ensuring that the lead is properly inserted and is in proper electrical contact with the components of the header of the IPG may be difficult for the clinician during the implant medical procedure.

In other designs, a stiffener component may be included within the proximal end of a stimulation lead to provide greater column strength. For example, a tube or similar component may be included within the polymer material of the stimulation lead and within the terminals of the lead to provide additional column strength. Nitinol or other suitable materials may be employed for such components. A variety of designs of stiffening components have been proposed including the designs in U.S. Pat. No. 8,244,372, the contents of which is incorporated herein by reference in its entirety.

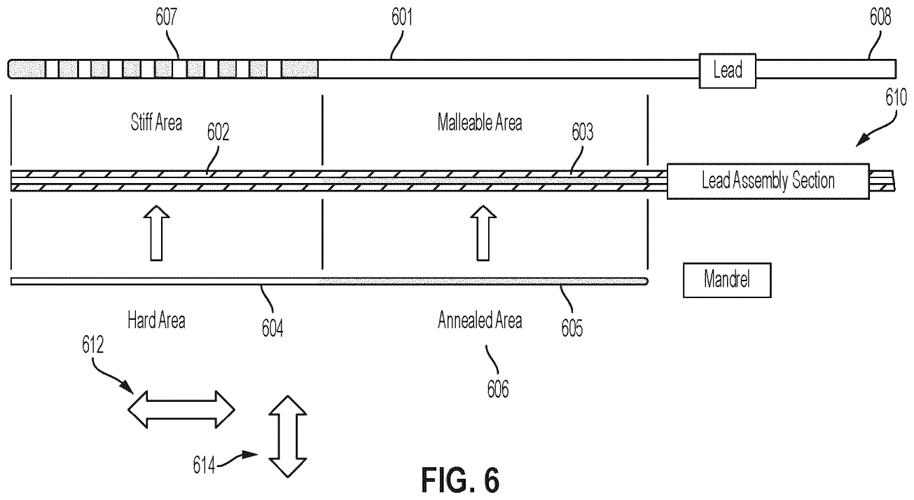

Referring to FIG. 5, the additional column strength assists with insertion of the proximal end of leads 501 into the header of the IPG. However, the transitioning between the stiff section of the proximal end of the lead to the flexible section of the lead body creates a mechanical issue. Specifically, a common technique employed by surgeons for an implant procedure involves coiling an excess lead length about the IPG in the implant pocket. The coil configuration creates a stress point, shown in region 502, within the stimulation lead where the stiffener component ends within the lead body. The stress point(s) may cause lead failure over time by repeated flexing due to bodily movement of the patient.

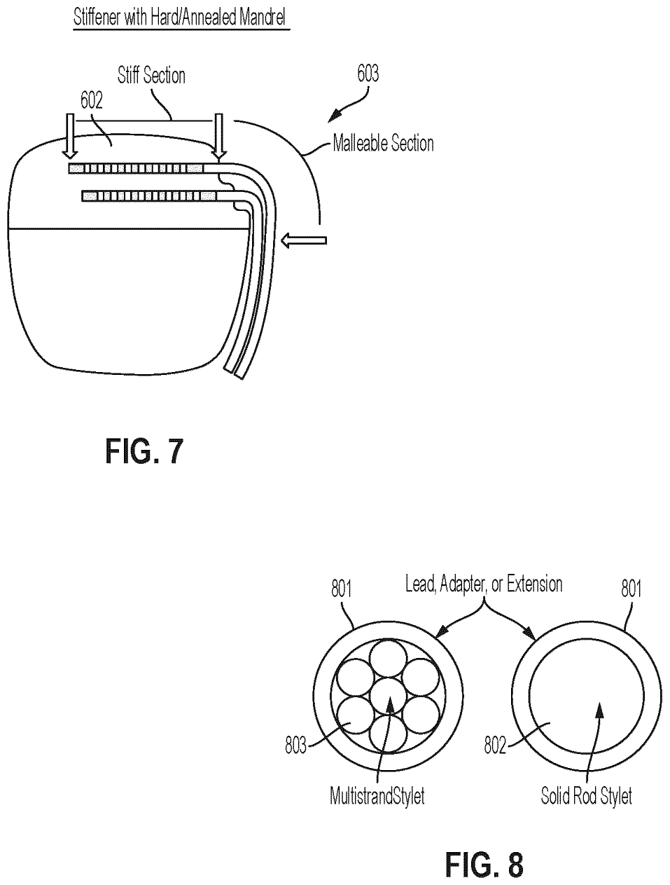

Referring to FIG. 6, a diagram illustrating aspects of a lead for use within neurostimulation systems in accordance with the present disclosure is shown as a lead 601. Lead 601 includes flexible section 608. Flexible section 608 possesses minimal column strength provided by the polymer material of the lead body of stimulation lead 601. Further, lead 601 includes a stiffener component 606 (e.g., a mandrel structure) to assist with insertion of the terminals 607 into the header of an IPG (e.g., the IPG 150 of FIG. 1). As shown in internal view 610 of FIG. 6, the lead 601 also includes a stiff section 602 and malleable section 603. The stiff section 602 largely extends along the region with terminals 607 and, upon insertion, will be contained within the header of the IPG. The malleable section 603 is immediately adjacent to stiff section 603 and is disposed just past electrodes 607. Malleable section 603 provides a transition between the stiff section 602 and flexible section 608. Malleable section 603 exhibits a greater degree of stiffness than flexible portion 608. However, malleable section 603 is more flexible than stiff section 602 and malleable section 603 can be shaped by clinician.

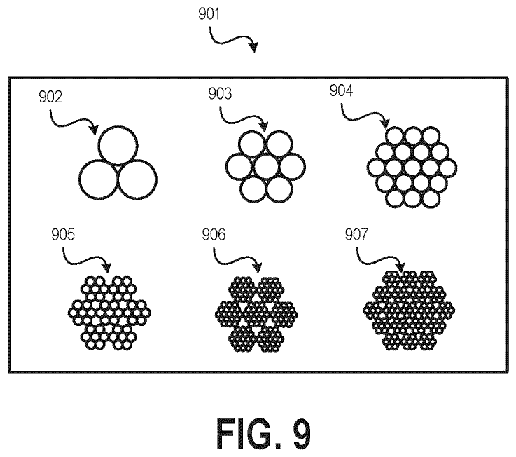

As shown in FIG. 6, the stiff section 602 and malleable section 603 are defined by mandrel 606. In an aspect, the mandrel 606 may be fabricated by subjecting suitable implantable metal material to an annealing process. Specifically, hard area 604 is not subjected to annealing while annealed area 605 is annealed before inclusion within lead 601. The annealing process reduces the stiffness of mandrel 606 but allows mandrel 606 within the annealed area 605 to retain some degree of mechanical integrity. Annealed area 605 may be bent or otherwise shaped and, once so modified, will retain its modified shaped. Accordingly, upon implantation, a surgeon may coil lead 601 in the implant pocket, which may include modifying the shape of malleable section 603. For example, as shown in FIG. 7, the malleable section 603 may be shaped to conform to the external surface of the IPG, thereby avoiding the stress points exhibited by traditional lead arrangements, as described above with reference to FIG. 5, and minimizing the likelihood that a lead failure occurs once the lead and IPG are implanted within the implant pocket and increasing the comfort to the patient. Thus, malleable section 603 provides a lead design configuration that prevents creation of stress points in the manner exhibited by other stiffener designs, while hard area 604 enables the proximal portion of the lead to be fully inserted into the IPG, thereby creating good electrical contact between the electrodes and the header.

As previously discussed, the main portion of a stimulation lead is generally flexible and exhibits minimal column strength. A stylet is commonly used as a stiffener when the lead is delivered to the anatomical location. It is inserted into the lead inner lumen during placement and is extracted after the lead is placed at the targeted location and the lead body is formed to loops allowing for ideal strain relief. A stylet not only needs to be stiff enough to provide column strength to help drive the lead to the anatomical location but also needs to be flexible so it can help form a loop of the lead.

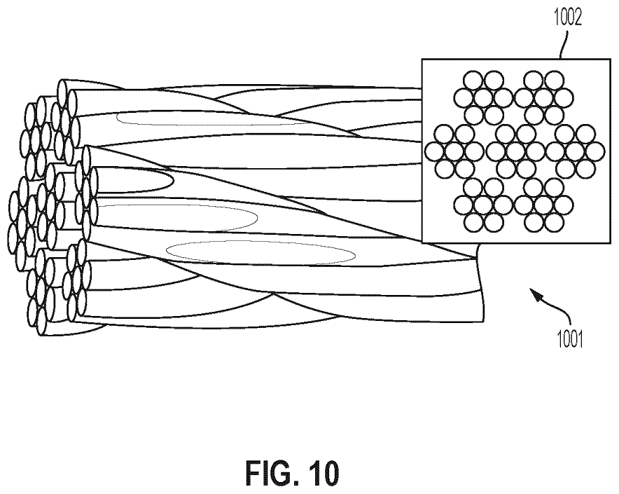

Referring to FIG. 8, cross-sectional views comparing a stranded stylet in accordance with aspects of the present disclosure to a solid rod stylet are shown. As shown in FIG. 8, conventional stylets use a rod 802 that may be inserted into the lead inner lumen of a lead 801. In contrast to such a design, a stylet 802 according to the present disclosure may be formed using one or more stranded cables. The stylet 802 provides a design that is both highly flexible yet capable of providing superior column strength for easy insertion and easy lead deployment. The primary advantage of a stranded stiffener or stylet is its ability to provide both column strength and flexibility. When pushed or compressed along its main axis (e.g., the directions shown by arrow 612 of FIG. 6), such as when inserting a lead into an IPG, stylet 803 formed from stranded cable provides sufficient column strength to allow for easy insertion. However, the stylet 803, when loaded laterally (e.g., the directions shown by arrow 614 of FIG. 6), such as when wrapping the lead around the IPG for implantation, can be highly flexible and provide little reaction force upon the patients tissue after the lead is implanted. Additionally, the highly flexible nature of a stranded cable enables the cables to be flexed significantly and repeatedly without breaking, unlike rods and tubes.

Given the nature of the multistrand construction for the stylet 803, the contact area between the multistrand stylet 803 outer surface and the surface of the lead inner lumen (see inner surface of lead 801 in FIG. 8) is smaller as compared to the solid rod stylet 802 (assuming the same outer diameter). The reduced contact area of the multistrand stylet 803 significantly reduces the friction between two surfaces (i.e., the surface of the lead inner lumen and the exterior surface of the stylet) which allows for easy extraction of stylet 803 after placement of the lead at the targeted location.

Stranded cable is comprised of multiple individual smaller strands referred to as filars, that are held together, typically by twisting them about their long axis. The filar material, the filar diameter, the number of filars, how aggressively the filars are twisted together (called the lay length), and the direction of twisting are used to control the column strength to stiffness of the overall cable. The basic form of stylets according to some embodiments, utilizes a stranded cable cut to a specific length that is inserted in place of the current hypo-tube or rod used today. FIG. 9 shows block diagrams depicting exemplary filar designs 901 according to some representative embodiments. As can be appreciated from FIG. 9, the exemplary filar designs 901 include example designs 902, 903, 904, 905, 906, 907, each having a respective different number of filars. For example, design 902 includes 3 filars, design 903 includes 7 filars, and design 904 includes 19 filars. Furthermore and as shown in FIG. 9, some filar designs may be formed using multiple composite filars (e.g., filars formed from multiple smaller filars). For example, design 905 illustrates a filar design which is formed from 7 groups of filars each configured according to design 903, design 906 illustrates a filar design which is formed from 7 groups of filars each configured according to design 904, and design 907 illustrates a filar design which is formed from 19 groups of filars each configured according to design 903. It is noted that composite filars may be formed from groups of filars having smaller diameters than if non-composite filars are used. For example, a stylet formed according to filar design 903 may have larger diameter filars as compared to individual filars of the composite filar designs 905, 907. Referring to FIG. 10, an image and diagram depicting a filar design 1001 in further detail according to some representative embodiments is shown. As shown in inset 1002, the filar design 1001 is configured according to the composite filar design 905. It is noted that the exemplary filar designs of FIGS. 9 and 10 have been provided for purposes of illustration, rather than by way of limitation and that other filar designs (or composite filar designs) may be utilized by embodiments of the present disclosure.

In addition to the design properties listed above, the column strength of the stranded cable can be increased by compressing it. The compression process is essentially a swaging process, where the outer diameter of the final cable is forcibly compressed increasing the area-moment of the design. This process also allows for a precise control of the outer diameter of the stiffener or stylet. As can be appreciated from the foregoing, by adjusting material, filar diameter, lay length, compression amount and even the number of filars included in the design, the stranded cable can be customized for a wide variety of stiffness and flexibility property requirements, as well as provide a tradeoff between these properties.

Additionally, the stranded cable can be improved by welding the ends of the stranded stiffener together to ensure that the strands remain twisted together and to avoid an individual filar from protruding out the soft body of the lead extension, or adapter. The welded end also ensures the end the cable does not damage the lead body as well as makes it easier to assemble and possibly insert into the stylet of the lead body.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112 (f), unless and until such claim limitations expressly use the phrase “means for” followed by a statement of function void of further structure.

1. An implantable stimulation lead for applying electrical pulses to a patient, the implantable stimulation lead comprising:
a plurality of conductors for conducting electrical pulses;
a lead body of insulating material enclosing the plurality of conductors;
a plurality of electrodes, on a distal portion of the stimulation lead, that are coupled to the plurality of conductors, wherein the plurality of electrodes are adapted to apply electrical pulses to tissue of the patient;
a plurality of terminals, on a proximal portion of the stimulation, that are coupled to the plurality of conductors, wherein the plurality of terminals are adapted to receive electrical pulses from an implantable pulse generator;
a stiffening component within the lead body at a proximal portion of the stimulation lead, wherein the stiffening component comprises an annealed section and a non-annealed section, wherein the non-annealed section is adapted to maintain a substantially linear configuration for insertion of the plurality of terminals within a header of an implantable pulse generator, and wherein the annealed section is malleable to be shaped by a clinician into a desired curved or partially coiled configuration within a lead implant pocket location.

2. The implantable stimulation lead of claim 1, wherein the non-annealed section is configured to be contained within the header of the implantable pulse generator after insertion.

3. The implantable stimulation lead of claim 1, wherein the stiffening component is formed from a metal material.

4. The implantable stimulation lead of claim 3, wherein the annealed section corresponds to a first portion of the metal material and the non-annealed portion corresponds to a second portion of the metal material.

5. The implantable stimulation lead of claim 1, wherein the annealed portion is configured to maintain a shape corresponding to the curved or partially coiled configuration once shaped by the clinician.

6. The implantable stimulation lead of claim 5, wherein the non-annealed portion has a column strength configured for driving the stiffening component into an anatomical location.

7. The implantable stimulation lead of claim 1, wherein the stiffening component comprises a stranded metal cable.

8. The implantable stimulation lead of claim 7, wherein the annealed portion of the stranded metal cable exhibits flexibility when loaded laterally to support the desired curved or partially coiled configuration.

9. The implantable stimulation lead of claim 7, wherein the stranded metal cable comprises a plurality of filars.

10. The implantable stimulation lead of claim 7, wherein the plurality of filars are twisted about a longitudinal axis of the stranded metal cable.

11. The implantable stimulation lead of claim 10, wherein the plurality of filars have a composite configuration.

12. A neurostimulation system for applying electrical pulses to neural tissue of a patient, the neurostimulation system comprising:
the implantable pulse generator; and
one or more stimulation leads according to claim 1.

13. A device kit for an implantation procedure for a neurostimulation patient, the kit comprising:
an implantable stimulation lead for applying electrical pulses to a patient, comprising: (1) a plurality of conductors for conducting electrical pulses; (2) a lead body of insulating material enclosing the plurality of conductors; (3) a plurality of electrodes, on a distal portion of the stimulation lead, that are coupled to the plurality of conductors, wherein the plurality of electrodes are adapted to apply electrical pulses to tissue of the patient; (4) a plurality of terminals, on a proximal portion of the stimulation, that are coupled to the plurality of conductors, wherein the plurality of terminals are adapted to receive electrical pulses from an implantable pulse generator; and (5) a lumen to receive a stylet;
a stylet to guide the stimulation lead during the implantation procedure, wherein the stylet comprises multiple strands of filars twisted together according to a lay length to provide column strength to advance the stimulation lead during implantation while exhibiting flexibility out of plane relative to column strength for advancement of the stimulation lead.

14. The device kit of claim 13, wherein the multiple strands of filars form a stranded metal cable.

15. The device kit of claim 14, wherein the multiple strands of filars are twisted about a longitudinal axis of the stranded metal cable.

16. The device kit of claim 15, wherein the stranded metal cable comprises filars having a composite configuration.

17. The device kit of claim 15, wherein the composite configuration comprises multiple groups of filars, each group of filars of the multiple groups of filars comprising a subset of the multiple filars.

18. The device kit of claim 17, wherein the multiple groups of filars are twisted together about a longitudinal axis of the stranded metal cable.

19. The device kit of claim 14, wherein the stranded metal cable comprises an annealed portion and a non-annealed section.

20. The device kit of claim 14, wherein the multiple filars are welded together at a distal end of the stranded metal cable, a proximal end of the stranded metal cable, or both.
